SEQUENCE LISTING (9 sequences)

SEQ ID NO: 1 - DNA, 1566 bp, synthetic construct
  source 1..1566: mol_type = other DNA
tgaagtttaaaatataagttataataaaaaatatatatatgatacgttggagatggcccctacaagtcgtaaaggccctggcccccaccgttgcttccagtaggtcatgatgcagcgcacctgtcctcccgcgctgctccaccccttcccactcctcccatttgcaagtcccagtaccaggttttcaaaaccgccttcacgcagtccacagataaaataccggcctcctcctccctttccccctccgattcccgcgcccgcccctacgctgccaaaagcgggggacaccgcccgtccccctccccgtccgggtctccacaagcgcacctgcgcgcgcgcgcccggcttccgttgcgcagcagggcagctgggcagggcagggcaggtcgccatttagtcctctgcgctttttggcgttggttcttcttgcacaactcttctgctggattccagctctgtttttctttctcctgtcctgctggtcagtcaaatatatcctcctggtcctgggtagcttttgggttccggcgccttgccgttcgcagtttgtgttcttggtcatctgatctgggtggtattaatctggagacccagtcactcggtcgggacgagatcaataaaaaggaagaaagaagcagcgcggcaagccggcggcgtcgcgaagcttaagatcagaggtttctaccttttttttcttcaagtgtaacgtaacctttgtaatctggatgcgttcaccgtaaatcctgattatgtcgagcggttcttgatttctttctcgtcttcgctgcatgttgagcagacaccgttggttgggcgttgcttcagatataatctacaagaccaagctcagattccatcagtcggtacgtacgtcggaacagcctcacttcgctcgctgctgcgcgttagatgccggttgcttcgtcgctaatggcgatggagttggagacggaccaactcgcctgggcggagcagcagcggcagcagaataggaggcagaccatggtcgtctgcagaaagagcgacgcagcggtggccaaagggcagcagcgtcagaacgcttcgccgccgtcgcccaagcctccgcccgcgggcgggctcagcgcggaggcgttcttggttctggcgtgcgtcgccgtgtcgctcatcgtgctgccgctggtcctgccgccgctgtcgcccccgccgcctctgctgctgctggtgccggtgtgcctgctcctgctcctcgccgcgctcgccaccttcgtgccgtcggatgtcaggagcatgccatcctccaacttgtaactactagttgttttgctagtcaatatccataaattctttttgttcaagtcggcatttatgtctgtgcatatggcataaaatgagtgtaatgaaatggaaatcttgtcttatctttctttttttggcaaacagacgttcaccgttagatcgaacaaactactacgtacttgtgctttctgcctgcgttttggttgaattcatctagtttctactggtttaaccaaattatatattattatgtatttgtactaacaacacgtttagtttaacatataa

SEQ ID NO: 2 - DNA, 381 bp, synthetic construct
  source 1..381: mol_type = other DNA
atgccggttgcttcgtcgctaatggcgatggagttggagacggaccaactcgcctgggcggagcagcagcggcagcagaataggaggcagaccatggtcgtctgcagaaagagcgacgcagcggtggccaaagggcagcagcgtcagaacgcttcgccgccgtcgcccaagcctccgcccgcgggcgggctcagcgcggaggcgttcttggttctggcgtgcgtcgccgtgtcgctcatcgtgctgccgctggtcctgccgccgctgtcgcccccgccgcctctgctgctgctggtgccggtgtgcctgctcctgctcctcgccgcgctcgccaccttcgtgccgtcggatgtcaggagcatgccatcctccaacttgtaa

SEQ ID NO: 3 - protein, 126 aa, synthetic construct
MPVASSLMAMELETDQLAWAEQQRQQNRRQTMVVCRKSDAAVAKGQQRQNASPPSPKPPPAGGLSAEAFLVLACVAVSLIVLPLVLPPLSPPPPLLLLVPVCLLLLLAALATFVPSDVRSMPSSNL

SEQ ID NO: 4 - DNA, 20 bp, synthetic construct
  source 1..20: mol_type = other DNA
atgccggttgcttcgtcgct

SEQ ID NO: 5 - DNA, 23 bp, synthetic construct
  source 1..23: mol_type = other DNA
gtagttacaagttggaggatggc

SEQ ID NO: 6 - DNA, 38 bp, synthetic construct
  source 1..38: mol_type = other DNA
tgcaggtcgactctagagatgccggttgcttcgtcgct

SEQ ID NO: 7 - DNA, 39 bp, synthetic construct
  source 1..39: mol_type = other DNA
ggtctttgtagtccatttgcaagttggaggatggcatgc

SEQ ID NO: 8 - DNA, 23 bp, synthetic construct
  source 1..23: mol_type = other DNA
gacggaccaactcgcctgggcgg

SEQ ID NO: 9 - DNA, 23 bp, synthetic construct
  source 1..23: mol_type = other DNA
ctgcagaaagagcgacgcagcgg

(19) United States
(12) Patent Application Publication
(10) Pub. No.: US 2024/0401072 A1
(43) Pub. Date: Dec. 5, 2024

(54) METHOD FOR USING ZMARGOS9 GENE IN DROUGHT RESISTANCE AND HIGH YIELD PRODUCTION OF MAIZE

(71) Applicant: Sichuan Agricultural University, Chengdu (CN)

(72) Inventors: Xuanjun FENG, Chengdu (CN); Yanli LU, Chengdu (CN); Huarui GUAN, Chengdu (CN); Weixiao ZHANG, Chengdu (CN); Xuemei ZHANG, Chengdu (CN); Qingjun WANG, Chengdu (CN); Yaxi LIU, Chengdu (CN); Jie XU, Chengdu (CN); Fengkai WU, Chengdu (CN)

(21) Appl. No.: 18/665,653

(22) Filed: May 16, 2024

(30) Foreign Application Priority Data
Jun. 2, 2023 (CN) 202310645709.6

(51) Int. Cl. C12N 15/82

(52) U.S. Cl. CPC C12N 15/8273; C12N 15/8261

(57) ABSTRACT

The present embodiments of the invention disclose methods for implementing ZmARGOS9 gene in drought resistance and high yield of maize. ZmARGOS9 is overexpressed in maize in the present disclosure. The high expression of ZmARGOS9 gene improves the drought resistance and yield of maize. Accordingly, it enables enrichment of drought-resistant and high-yielding genes, contributing to the security of the seed industry.

### CROSS-REFERENCE TO RELATED APPLICATIONS

This application claims priority to Chinese Patent Application No. 202310645709.6 filed on Jun. 2, 2023, the contents of which are hereby incorporated by reference.

### INCORPORATION BY REFERENCE STATEMENT

This statement, made under Rules 77 (b) (5) (ii) and any other applicable rule incorporates into the present specification of an XML file for a “Sequence Listing XML” (see Rule 831 (a)), submitted via the USPTO patent electronic filing system or on one or more read-only optical discs (see Rule 1.52 (e) (8)), identifying the names of each file, the date of creation of each file, and the size of each file in bytes as follows:
File name: 347091_12486
Creation date: Nov. 1, 2023
Byte size: 10,919

### TECHNICAL FIELD

The present disclosure relates to the field of biotechnology, and in particular to method for using ZmARGOS9 gene in drought resistance and high yield production of maize.

### BACKGROUND

Drought looms large as a threat to food production as the global warming worsens and drought incidence becomes more frequent. Efforts have been done by breeders to improve crop drought resistance through genetic manipulation. However, it is difficult in most cases to manipulate a gene to enhance drought resistance without compromising yield under normal conditions. Nelson et al., over-expressed the NF-YB2 gene in maize to improve maize yield under drought, and Kudo et al. used a combination of drought-resistant genes and growth-promoting genes to improve plant drought tolerance under drought and to promote plant growth under normal conditions. Feng Xuanjun et al. used drought-induced expression of the ZmDRO1 gene to achieve improved drought tolerance in transgenic maize without affecting yield under normal conditions. The TPP (trehalose-6-phosphate phosphatase) gene and the bacteria Csp (cold shock proteins) gene have been successfully used in breeding for drought tolerance of maize. Heterosis is one of the most important factors in determining maize yield, and taking full advantage of the hybrid dominance can significantly increase maize yield. The ARGOS (also called ZAR) gene has been reported in several studies to promote plant growth by promoting cell division or cell elongation, and it is probably an important regulatory gene of heterosis. Shi et al. reported that overexpression of ZmARGOS1 enhances drought resistance of maize, and successfully increased drought resistance of maize without affecting agronomic traits under normal conditions by root-specific high expression of ZmARGOS8. The growth period is also an ongoing concern for breeders. Without causing crop yield reduction, an appropriate shortening of the growth period can reduce management costs, and may also reduce the risk of yield reduction caused by occasional natural disasters. Therefore, short growth period (early maturity) is also an important indicator for breeders in selecting good varieties.

Balancing yield and resistance are major challenges for breeders. Overexpression of the NF-YB2 gene increases the yield of transgenic maize under drought stress, but agronomic traits under normal conditions have not been studied in detail. zmDRO1 may inhibit plant growth in case of high expression caused by environmental influences. Overexpression of ZmARGOS1 reduces plant yield under normal conditions, and overexpression of ZmARGOS8 causes plants to be too tall and prone to collapse, so that the investigators used a root-specific expression of maize. The genes TPP, CspA and CspB, which have been successfully applied commercially by Monsanto Company, can improve drought tolerance in transgenic plants without compromising yield under normal conditions. However, the intellectual property rights for the application of these genes do not belong to the Chinese researchers, and the relevant genes are not available for use by Chinese breeding companies or require costly acquisition of patent rights. Therefore, there is a strong demand for drought-resistant genes with independent intellectual property rights. As it is not often reported that a single gene can improve both crop resistance and yield, such a genetic resource is very precious and has a high value for production use.

### SUMMARY

It is an objective of the present disclosure to provide a method for using ZmARGOS9 gene in drought resistance and high yield of maize, so as to solve the problems existing in the above mentioned prior art. According to the embodiments of the present disclosure, provides advantages of high expression of ZmARGOS9 gene that improves the drought resistance and yield of maize, therefore, it enables enrichment of drought-resistant and high-yielding genes, contributing to the security of the seed industry.

In order to achieve the above objectives, the present disclosure provides following technical schemes:

The present disclosure provides a method for improving drought resistance of maize by implementing any one of the following substances (i through iv):
(i) firstly, it comprises a protein, where the protein has an amino acid sequence as shown in SEQ ID No. 3;
(ii) secondly, it comprises a ZmARGOS9 gene, where the ZmARGOS9 gene has a nucleotide sequence as shown in SEQ ID No. 2;
(iii) thirdly, it comprises a recombinant plasmid, including the ZmARGOS9 gene; and
(iv) fourthly, it comprises a recombinant microbial strain, including the recombinant plasmid.

The present disclosure also provides a method for improving maize yield by implementing any one of the following substances (i through iv):
(i) firstly, it comprises a protein, where the protein has an amino acid sequence as shown in SEQ ID No. 3;
(ii) secondly, it comprises a ZmARGOS9 gene, where the ZmARGOS9 gene has a nucleotide sequence as shown in SEQ ID No. 2;
(iii) thirdly, it comprises a recombinant plasmid, including the ZmARGOS9 gene; and
(iv) fourthly, it comprises a recombinant microbial strain, including the recombinant plasmid.

The present disclosure also provides a method for shortening a growth period of maize by implementing any one of the following substances (i through iv):
(i) firstly, it comprises a protein, where the protein has an amino acid sequence as shown in SEQ ID No. 3;
(ii) secondly, it comprises a ZmARGOS9 gene, where the ZmARGOS9 gene has a nucleotide sequence as shown in SEQ ID No. 2;
(iii) thirdly, it comprises a recombinant plasmid, including the ZmARGOS9 gene; and
(iv) fourthly, it comprises a recombinant microbial strain, including the recombinant plasmid.

The present disclosure also provides a method for improving drought resistance of maize, including steps of:
transforming a ZmARGOS9 gene into maize plants, and constructing to obtain high expression plants of the ZmARGOS9 gene;
where the ZmARGOS9 gene has a nucleotide sequence as shown in SEQ ID No. 2.

The present disclosure also provides a method for improving maize yield, including steps of:
transforming a ZmARGOS9 gene into maize plants, and constructing to obtain high expression plants of the ZmARGOS9 gene;
where the ZmARGOS9 gene has a nucleotide sequence as shown in SEQ ID No. 2.

The present disclosure also provides a method for shortening a growth period of maize, including steps of:
transforming a ZmARGOS9 gene into maize plants, and constructing to obtain high expression plants of the ZmARGOS9 gene;
where the ZmARGOS9 gene has a nucleotide sequence as shown in SEQ ID No. 2.

The present disclosure has the following technical effects.

According to the present disclosure, the overexpression of ZmARGOS9 in maize reveals that the growth and development of transgenic plants are faster than that of the control starting from the germination stage, with increased yields and significantly enhanced drought tolerance. Thus, overexpression of ZmARGOS9 is capable of increasing the yield of maize under both drought conditions and normal conditions, in addition to appropriately shortening the growth period. The identification of ZmARGOS9 therefore enriches the drought-resistant and high-yielding genes in China and adds to the security of the seed industry.

### BRIEF DESCRIPTION OF THE DRAWINGS

For a clearer description of the technical schemes in the embodiments or prior art of the present disclosure, the accompanying drawings to be used in the embodiments are briefly described hereinafter, and it is obvious that the accompanying drawings in the description hereinafter are only some of the embodiments of the present disclosure, and that for the person of ordinary skill in the field, other accompanying drawings are available on the basis of the accompanying drawings without any creative labour.

FIG. 1 shows an expression cassette of plasmid pLANT-cFlag.

FIG. 2A shows phenotypes of each material at the germination stage.

FIG. 2B shows phenotypes of each material of 56 days.

FIG. 2C shows phenotypes of each material of 100 days.

FIG. 2D shows phenotypes of female ear after harvest.

FIG. 2E shows ear weight data after sun drying.

FIG. 2F shows ear grain weight after sun drying.

FIG. 3A shows tassel phenotypes of each material.

FIG. 3B shows numbers of branches of tassels.

FIG. 3C shows growing days at the staminate stage.

FIG. 3D shows growing days at a pollen dispersal stage.

FIG. 3E shows growing days at a silking stage.

FIG. 4A shows ear weight data of each material under drought conditions after sun drying.

FIG. 4B shows ear grain weight of each material under drought conditions.

### DETAILED DESCRIPTION OF THE EMBODIMENTS

A number of exemplary embodiments of the present disclosure are now be described in detail, and this detailed description should not be considered as a limitation of the present disclosure, but should be understood as a rather detailed description of certain aspects, characteristics and embodiments of the present disclosure.

It should be understood that the terminology used in the present disclosure is only for describing specific embodiments and is not used to limit the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges can be independently included or excluded from the range.

Unless otherwise specified, all technical and scientific terms used herein have the same meaning as commonly understood by one of ordinary skill in the art to which this present disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes can be made to the specific embodiments of the present disclosure without departing from the scope or spirit of the present disclosure. Other embodiments are apparent to the skilled person from the description of the invention. The description and embodiments of that present disclosure are exemplary only.

The terms “including”, “comprising”, “having” and “containing” used in this specification are all open terms, which means including but not limited to.

### Embodiment 1

1. DNA (Deoxyribonucleic acid) and protein characterization of ZmARGOS9

According to an embodiment of the present invention disclosure, the ZmARGOS9 gene may be derived from maize and has a common gene number GRMZM2G082943 (reference genomic information B73 Ref_v3) or Zm00001d041774 (reference genomic information B73 Ref_v4), the DNA. CDS (coding sequence) and protein sequences thereof are shown below, and the gene contains only one exon. Transgenic expression is performed using a fusion of the CDS thereof and Flag tag sequences in the present application.

>GRMZM2G082943_DNA
(SEQ ID No. 1)
TGAAGTTTAAAATATAAGTTATAATAAAAAATATATATATGATACGTTGG
AGATGGCCCCTACAAGTCGTAAAGGCCCTGGCCCCCACCGTTGCTTCCAG
TAGGTCATGATGCAGCGCACCTGTCCTCCCGCGCTGCTCCACCCCTTCCC
ACTCCTCCCATTTGCAAGTCCCAGTACCAGGTTTTCAAAACCGCCTTCAC
GCAGTCCACAGATAAAATACCGGCCTCCTCCTCCCTTTCCCCCTCCGATT
CCCGCGCCCGCCCCTACGCTGCCAAAAGCGGGGGACACCGCCCGTCCCCC
TCCCCGTCCGGGTCTCCACAAGCGCACCTGCGCGCGCGCGCCCGGCTTCC
GTTGCGCAGCAGGGCAGCTGGGCAGGGCAGGGCAGGTCGCCATTTAGTCC
TCTGCGCTTTTTGGCGTTGGTTCTTCTTGCACAACTCTTCTGCTGGATTC
CAGCTCTGTTTTTCTTTCTCCTGTCCTGCTGGTCAGTCAAATATATCCTC
CTGGTCCTGGGTAGCTTTTGGGTTCCGGCGCCTTGCCGTTCGCAGTTTGT
GTTCTTGGTCATCTGATCTGGTGGTATTAATCTGGAGACCCAGTCACTCG
GTCGGGACGAGATCAATAAAAAGGAAGAAAGAAGCAGCGCGGCAAGCCGG
CGGCGTCGCGAAGCTTAAGATCAGAGGTTTCTACCTTTTTTTTCTTCAAG
TGTAACGTAACCTTTGTAATCTGGATGCGTTCACCGTAAATCCTGATTAT
GTCGAGCGGTTCTTGATTTCTTTCTCGTCTTCGCTGCATGTTGAGCAGAC
ACCGTTGGTTGGGCGTTGCTTCAGATATAATCTACAAGACCAAGCTCAGA
TTCCATCAGTCGGTACGTACGTCGGAACAGCCTCACTTCGCTCGCTGCTG
CGCGTTAGATGCCGGTTGCTTCGTCGCTAATGGCGATGGAGTTGGAGACG
GACCAACTCGCCTGGGCGGAGCAGCAGCGGCAGCAGAATAGGAGGCAGAC
CATGGTCGTCTGCAGAAAGAGCGACGCAGCGGTGGCCAAAGGGCAGCAGC
GTCAGAACGCTTCGCCGCCGTCGCCCAAGCCTCCGCCCGCGGGCGGGCTC
AGCGCGGAGGCGTTCTTGGTTCTGGCGTGCGTCGCCGTGTCGCTCATCGT
GCTGCCGCTGGTCCTGCCGCCGCTGTCGCCCCCGCCGCCTCTGCTGCTGC
TGGTGCCGGTGTGCCTGCTCCTGCTCCTCGCCGCGCTCGCCACCTTCGTG
CCGTCGGATGTCAGGAGCATGCCATCCTCCAACTTGTAACTACTAGTTGT
TTTGCTAGTCAATATCCATAAATTCTTTTTGTTCAAGTCGGCATTTATGT
CTGTGCATATGGCATAAAATGAGTGTAATGAAATGGAAATCTTGTCTTAT
CTTTCTTTTTTTGGCAAACAGACGTTCACCGTTAGATCGAACAAACTACT
ACGTACTTGTGCTTTCTGCCTGCGTTTTGGTTGAATTCATCTAGTTTCTA
CTGGTTTAACCAAATTATATATTATTATGTATTTGTACTAACAACACGTT
TAGTTTAACATATAA.
>GRMZM2G082943_CDS
(SEQ ID No. 2)
ATGCCGGTTGCTTCGTCGCTAATGGCGATGGAGTTGGAGACGGACCAACT
CGCCTGGGCGGAGCAGCAGCGGCAGCAGAATAGGAGGCAGACCATGGTCG
TCTGCAGAAAGAGCGACGCAGCGGTGGCCAAAGGGCAGCAGCGTCAGAAC
GCTTCGCCGCCGTCGCCCAAGCCTCCGCCCGCGGGCGGGCTCAGCGCGGA
GGCGTTCTTGGTTCTGGCGTGCGTCGCCGTGTCGCTCATCGTGCTGCCGC
TGGTCCTGCCGCCGCTGTCGCCCCCGCCGCCTCTGCTGCTGCTGGTGCCG
GTGTGCCTGCTCCTGCTCCTCGCCGCGCTCGCCACCTTCGTGCCGTCGGA
TGTCAGGAGCATGCCATCCTCCAACTTGTAA.
>GRMZM2G082943_protein
(SEQ ID No. 3)
MPVASSLMAMELETDQLAWAEQQRQQNRRQTMVVCRKSDAAVAKGQQRQN
ASPPSPKPPPAGGLSAEAFLVLACVAVSLIVLPLVLPPLSPPPPLLLLVP
VCLLLLLAALATFVPSDVRSMPSSNL.

2. Cloning and transgenic expression of ZmARGOS9

The total RNA (Ribonucleic Acid) is extracted from the tassel of maize inbred line B73, and then cDNA (complementary DNA) is generated by reverse transcription. Thereafter, a protein coding region of the ZmARGOS9 gene is amplified by PCR (Polymerase Chain Reaction), and finally the expression plasmid pLANT-cFlag is cloned by homologous recombination.

2.1 RNA (Ribonucleic Acid) extraction

The plant total RNA isolation kit (RE-05014, FOREGENE) of Foregene Company is used for RNA extraction. All kit equipment for the experiment is washed with DEPC (diethyl pyrocarbonate) water and sterilized at 121° C. for 1 h. During the RNA extraction, the samples are kept at a low temperature of 4° C. The specific steps are explained as follows:
(1) about 200 mg of the fresh sample is taken into a 1.5 mL centrifuge tube after thoroughly grinding the sample under liquid nitrogen;
(2) 50 μL of PSL1 (a buffer in RNA isolation kit) reagent is taken from the kit and 10 μL of β-mercaptoethanol are mixed to form a PSL1 mixture. The mixture is then set aside;
(3) the PSL1 mixture prepared in the previous step is added to the sample in the centrifuge tube and quickly shaken and mixed, then left to stand for a duration of 5 minutes;
(4) the sample of the previous step is added with 100 μL of reagent PS (a buffer in RNA isolation kit), mixed by inverting, and is centrifuged at 12000 r/min for a duration of 3 minutes;
(5) the supernatant from the previous step is transferred to a DNA cleaning column and the column is discarded after centrifugation at 12000 r/min for a duration of 2 minutes;
(6) PSL2 (a buffer in RNA isolation kit) reagent of 1.5 times the volume of the liquid in the collection tube is added to the liquid, and the liquid is gently mixed and transferred to an RNA only column, centrifuged at 12000 r/min for a duration of 2 minutes and the supernatant is discarded;
(7) reagent PRW1 (a buffer in RNA isolation kit) of 500 μL is added to the RNA only column, followed by centrifugation at 12000 r/min for a duration of 2 minutes, and the supernatant is discarded;
(8) absolute ethanol of 700 μL is added to the RNA only column, followed by centrifugation at 12000 r/min for a duration of 2 minutes, and the supernatant is discarded;
(9) PRW2 (a buffer in RNA isolation kit) of 700 μL is added to the RNA only column, followed by centrifugation at 12000 r/min for a duration of 2 minutes, and the supernatant is discarded. This step is repeated once more;
(10) the RNA only column tube is centrifuged at 12000 r/min for a duration of 3 minutes to remove residual reagents; and
(11) the RNA only column is transferred to a new 1.5 mL centrifuge tube, 50 μL of RNAase-free water preheated at 65° C. is added dropwise to the centre of the membrane, and the adsorbent column is discarded after centrifugation at 12,000 r/min for a duration of 3 minutes, and the RNA at the bottom of the tube is collected.
   2.2 Reverse Transcription cDNA

Reverse transcription is completed by using a Prime ScriptTM RT Reagent Kit (RR 047A) from TAKARA Company, with specific steps as follows:
(1) genomic DNA is removed (reaction system: 5×gDNA Eraser Buffer 2 μL, gDNA Eraser 1 μL, Total RNA 1 μg, supplemented with RNase-free deionized water to a volume of 10 μL) and placed in a PCR instrument at 42° C. for a duration of 2 minutes;
(2) reverse transcription reaction (reaction system: 10 μL of RNA liquid with DNA removed, 1 μL of PrimeScript RT Enzyme mixI, 1 μL of reverse transcription primer Oligo dT, 4 μL of 5×PrimeScript buffer2, 4 μL of RNase-free deionized water), mixing and reacting at 37° C. for 20 min, treating at 85° C. for 10 s and storing at −20° C.

### 2.3 PCR Amplification

The coding region of ZmARGOS9 protein is obtained by PCR amplification.

Primers used are mentioned below:

ARG-F1:
(SEQ ID No. 4)
ATGCCGGTTGCTTCGTCGCT;
ARG-R1:
(SEQ ID NO. 5)
GTAGTTACAAGTTGGAGGATGGC.

Reaction system: 10 μL 2×PCR buffer, 4 μL 2 mM dNTPs, 10 μm forward and reverse primers 0.3 μL each, 0.4 μL cDNA template, 0.4 μL KOD FX Neo DNA polymerase, supplemented with deionized water to 20 μL.

Amplification parameters: pre-denaturation at 94° C. for 2 min, unwinding at 98° C. for 30 s, extension at 68° C. for 15 s, and amplification for 38 cycles.

### 2.4 Construction of Transgenic Expression Plasmid

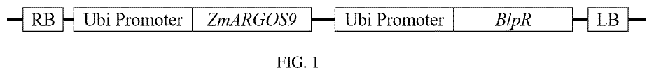

The coding region of ZmARGOS9 protein is connected to plant transgenic binary expression plasmid pLANT-cFlag by a homologous recombination cloning (see FIG. 1 for vector map information).

PCR amplification primers for homologous recombinant fragments are mentioned below:

ARG-F2:
(SEQ ID NO. 7)
TGCAGGTCGACTCTAGAGATGCCGGTTGCTTCGTCGCT;
ARG-R2:
(SEQ ID No. 6)
GGTCTTTGTAGTCCATTTGCAAGTTGGAGGATGGCATGC.

PCR amplification parameters and reaction system are the same as those in 2.3.

Enzyme digestion of vector: the binary expression plasmid pLANT-cFlag is digested by BamHI enzyme digestion site. Thereafter, the linearized plasmid is recovered by agarose gel electrophoresis, and the homologous recombination reaction is carried out by using ClonExpress Ultra One Step Cloning Kit (C115) of Novozen Company according to the instructions therein. After the constructed expression plasmid is confirmed to be correct by sequencing, Agrobacterium EHA105 is transformed, which is entrusted to Jiangsu Wimi Biotechnology Co., Ltd. by maize transformation to obtain transgenic maize with high expression of ZmARGOS9 gene, and the transgenic receptor material is KN5585.

### 3. Creation of ZmARGOS9 Gene Deletion Mutants

Jiangsu Wimi Biotechnology Co., Ltd. is entrusted to use CRISPR/Cas9 system to design double targets to edit the ZmARGOS9 gene; sequence of target 1: GACGGACCAACTCGCCTGGGCGG (SEQ ID No. 8); sequence of target 2: CTGCAGAAAGAGCGACGCAGCGG (SEQ ID No. 9). The resulting edited materials, KO1 and KO2, are deletions of large segments of the gene, resulting in the inability to encode the corresponding proteins.

### 4. Application of ZmARGOS9 in Drought Resistance and High Yield of Maize

### 4.1 Planting Experiment

KN5585, ZmARGOS9 gene deletion mutants (KO1 and KO2) and ZmARGOS9 high expression materials (OE1, OE2 and OE3) are planted in the field, with planting methods as follows:

The width of the field maize planting compartment is taken as 3.5 meters, the row spacing is taken as 0.8 meters, 10 holes are planted in each row, and 2 plants in each hole. The planting is carried out by seedling transplanting, where after seedling emergence in plug seedlings, diseased seedlings and weak seedlings are removed according to the situation, and seedlings are timely checked, supplemented and evened. Further, after careful soil preparation, transplanting is carried out at 2-3 leaf stage, and a small amount of compound fertilizer is applied during transplanting. Conventional field farming operations include weed control at seedling stage and topdressing at jointing stage. The mild drought treatment begins during the 12th leaf stage. If the drought is found to be more serious, the drought treatment group should be irrigated appropriately to supplement water. The experimental groups are watered normally according to their growth needs. Finally, the plants are subjected to natural pollination.

### 4.2 High Expression of ZmARGOS9 Promoting the Growth and Development of Maize and Increasing Yield

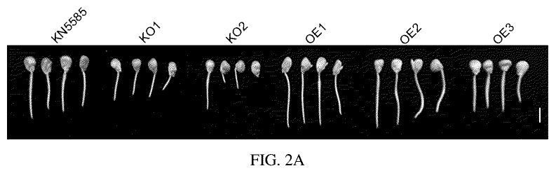

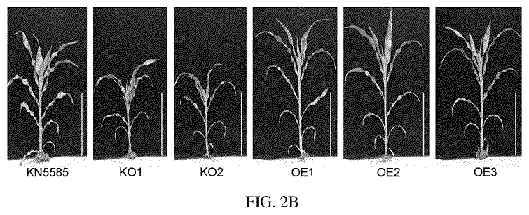

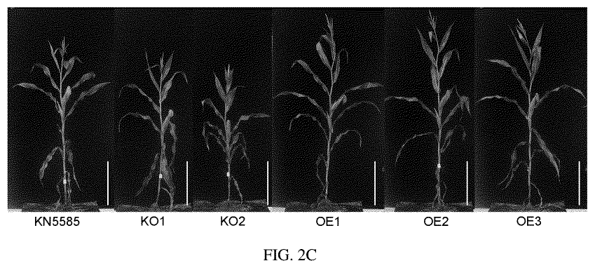

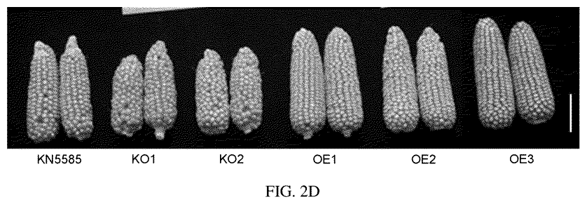

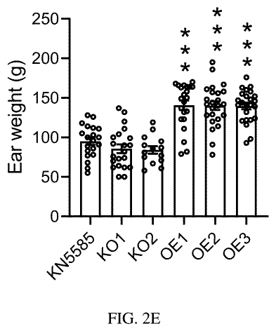

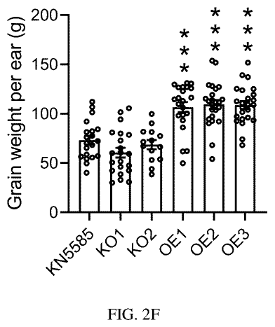

FIG. 2A-FIG. 2F shows the effect of ZmARGOS9 gene on the growth and yield of maize, where FIG. 2A shows phenotypes of each material at the germination stage, FIG. 2B shows phenotypes of each material of 56 days, FIG. 2C shows phenotypes of each material of 100 days, FIG. 2D shows phenotypes of female ear after harvest, FIG. 2E shows car weight data after sun drying, and FIG. 2F shows ear grain weight after sun drying. Among the figures, KN5585 is a transgenic negative material separated after transgenic operation, KO is a material with gene function deficiency; OE is a high expression material of ZmARGOS9 gene; the scales in FIG. 2A, FIG. 2B, FIG. 2C and FIG. 2D represents 1 cm, 50 cm, 50 cm and 5 cm respectively; significance of differences is analyzed using the student t-test, the asterisk (*) indicates that the material is significantly different from the wild type, where*indicates that it is significant at the level of P<0.05, **indicates that it is significant at the level of P<0.01 and ***indicates that it is significant at the level of P<0.001.

High expression of the ZmARGOS9 gene promotes the growth of seeds right at the seed germination stage, whereas deletion mutants of this gene shows a much slower germination rate (FIG. 2A). The plant height of high-expression materials is higher than that of wild type at 56 days and 100 days after germination, while the plant height of mutants is lower than that of wild type (FIG. 2B and FIG. 2C). The final spikelet weight and grain weight per spike of the high expression material are significantly greater than those of the wild type, whereas those of the mutant are smaller than those of the wild type (FIG. 2D-FIG. 2F).

### 4.3 High Expression of ZmARGOS9 Promoting Plant Development

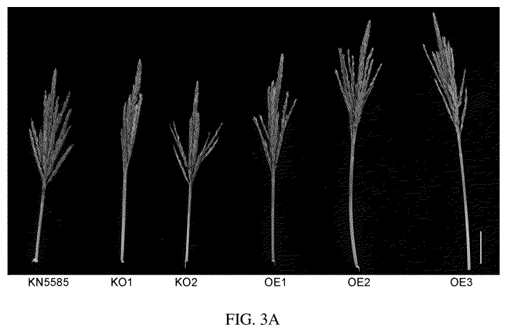

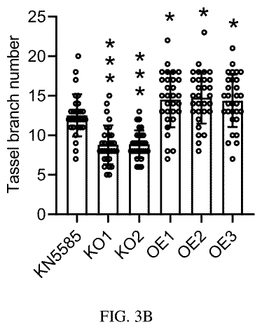

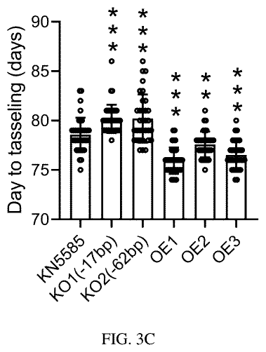

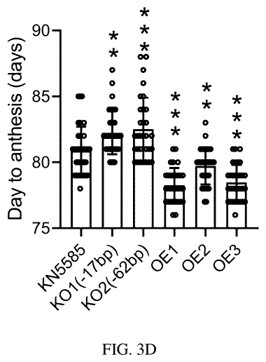

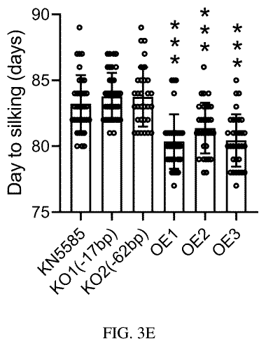

FIG. 3A-FIG. 3E illustrates the effect of ZmARGOS9 gene on maize tassel development, where FIG. 3A shows tassel phenotypes of each material, FIG. 3B shows numbers of branches of tassels, FIG. 3C shows growing days at the staminate stage, FIG. 3D shows growing days at the pollen dispersal stage, and FIG. 3E shows growing days at the silking stage; in the figures, KN5585 is a wild-type receptor material, KO is a material with gene function deficiency; OE is a high expression material of ZmARGOS9 gene; the scale in FIG. 3A represents 5 cm; significance of differences is analyzed using the student t-test, the*indicates that the material is significantly different from the wild type, where*indicates that it is significant at the level of P<0.05, **indicates that it is significant at the level of P<0.01 and ***indicates that it is significant at the level of P<0.001.

The tassel branches of materials with high expression of ZmARGOS9 gene are more than those of wild type, while those of mutants are less than those of wild type (FIG. 3A and FIG. 3B). Moreover, the growth period of the materials with high gene expression is slightly shorter than that of the wild type, and the tasseling, pollen dispersal and silking periods of the materials with high gene expression are all about 3 days shorter than those of the wild type, while the tasseling, pollen dispersal and silking periods of the mutant materials are delayed by 1-2 days (FIG. 3C-FIG. 3E).

### 4.4 High Expression of ZmARGOS9 Enhancing Drought Resistance of Maize

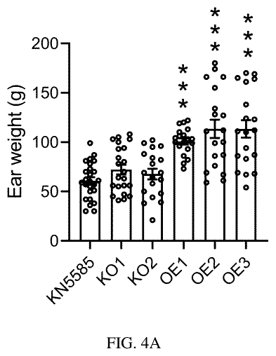

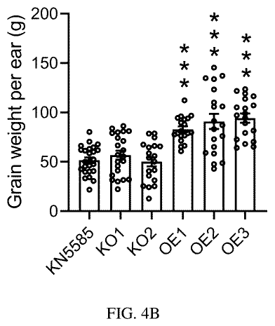

FIG. 4A and FIG. 4B illustrates the effect of ZmARGOS9 gene on drought tolerance of maize, where FIG. 4A shows ear weight data of each material under drought conditions after sun drying, and FIG. 4B shows car grain weight data of each material under drought conditions after sun drying. In these figures, KN5585 is a wild-type receptor material, KO is a material with gene function deficiency; OE is a high expression material of ZmARGOS9 gene; significance of differences is analyzed using the student t-test, the*indicates that the material is significantly different from the wild type, where*indicates that it is significant at the level of P<0.05, **indicates that it is significant at the level of P<0.01 and ***indicates that it is significant at the level of P<0.001.

Under mild drought conditions in the field, the spikelet weight and grain weight per spike of the ZmARGOS9 gene high expression material are still significantly higher than those of the wild type, whereas the difference between the mutant material and the wild type is not significant (FIG. 4A and FIG. 4B).

In accordance to the above, there is provided a method for improving drought resistance of maize, comprising steps of transforming ZmARGOS9 gene into maize plants, and constructing to obtain high expression plants of the ZmARGOS9 gene; where the ZmARGOS9 gene has a nucleotide sequence as shown in SEQ ID NO.2.

In another embodiment, a method for improving maize yield is disclosed. The method comprises the steps of transforming ZmARGOS9 gene into maize plants, and constructing to obtain high expression plants of the ZmARGOS9 gene, where the ZmARGOS9 gene has a nucleotide sequence as shown in SEQ ID NO.2.

In yet another embodiment, a method for shortening a growth period of maize is disclosed. The method comprises the steps of transforming ZmARGOS9 gene into maize plants, and constructing to obtain high expression plants of the ZmARGOS9 gene, where the ZmARGOS9 gene has a nucleotide sequence as shown in SEQ ID NO.2.

The above-mentioned embodiments only describe the preferred mode of the present disclosure, and do not limit the scope of the present disclosure. Under the premise of not departing from the design spirit of the present disclosure, various modifications and improvements made by ordinary technicians in the field to the technical scheme of the present disclosure shall fall within the protection scope determined by the claims of the disclosure.

What is claimed is:

1. A method for improving drought resistance of maize by implementing any one of following substances (i)-(iv):
(i) a protein, wherein the protein has an amino acid sequence as shown in SEQ ID NO.3;
(ii) a ZmARGOS9 gene, wherein the ZmARGOS9 gene has a nucleotide sequence as shown in SEQ ID NO.2;
(iii) a recombinant plasmid, comprising the ZmARGOS9 gene; and
(iv) a recombinant microbial strain, comprising the recombinant plasmid.

2. A method for improving maize yield by implementing of any one of following substances (i)-(iv):
(i) a protein, wherein the protein has an amino acid sequence as shown in SEQ ID NO.3;
(ii) a ZmARGOS9 gene, wherein the ZmARGOS9 gene has a nucleotide sequence as shown in SEQ ID NO.2;
(iii) a recombinant plasmid, comprising the ZmARGOS9 gene; and
(iv) a recombinant microbial strain, comprising the recombinant plasmid.

3. A method for shortening a growth period of maize by implementing any one of following substances (i)-(iv):
(i) a protein, wherein the protein has an amino acid sequence as shown in SEQ ID NO.3;
(ii) a ZmARGOS9 gene, wherein the ZmARGOS9 gene has a nucleotide sequence as shown in SEQ ID NO.2;
(iii) a recombinant plasmid, comprising the ZmARGOS9 gene; and
(iv) a recombinant microbial strain, comprising the recombinant plasmid.
